(19) United States
(12) Patent Application Publication
(10) Pub. No.: US 2013/0085418 A1
(43) Pub. Date: Apr. 4, 2013

(54) Device and Method for Monitoring a Patient Position

(71) Applicant: Khalil Salhani, Farnham (CA)

(72) Inventor: Khalil Salhani, Farnham (CA)

(21) Appl. No.: 13/630,844

(22) Filed: Sep. 28, 2012

(30) Foreign Application Priority Data
Sep. 29, 2011 (CA) 2754545

(51) Int. Cl. A61B 5/11; A61B 5/00

(52) U.S. Cl. CPC A61B 5/1113; A61B 5/746; A61B 5/7405; A61B 5/0002; A61B 5/7475
USPC 600/595

(57) ABSTRACT

A portable position safety device and method for the monitoring of a patient's position in a number of different situations are disclosed. The device comprises a gyroscope module, a processing unit and an alarm system. The device and method are designed to monitor the patient movement. As such, a movement of the patient's leg beyond a certain preset angle limit will trigger the device alarm system and alert the attendant. The monitoring of the patient's position is achieved using the gyroscope function.

### CROSS-REFERENCE TO RELATED APPLICATIONS

The present application is related o Canadian patent application no. 2,754,545.

### FIELD OF THE INVENTION

This invention generally relates to devices and methods for monitoring patient's activity. More particularly, this invention relates to devices and methods for notifying an attendant of a patient movement.

### BACKGROUND OF THE INVENTION

Patient activity monitoring devices of limited capability have been in use for some time. The most common type of patient activity monitoring system makes use of pressure sensitive electric switches which are responsive to the weight of the patient to indicate whether the patient has remained in bed. For example, U.S. Pat. No. 4,263,586 to Nicholas and U.S. Pat. No. 2,425,790 to Fletcher disclose the use of a pressure-operated electric switch which is installed under a leg of a bed to produce a signal in response to the lessening of the weight on the leg when the patient leaves the bed.

U.S. Pat. No. 3,325,799 to Farris and U.S. Pat. No. 2.260,715 to Ketchem both describe the use of a pressure operated electric switch which is installed beneath or within the mattress of the bed.

U.S. Pat. No. 4,067,005 to Levy, et al.; U.S. Pat. No. 3,961,201 to Rosenthal; and U.S. Pat. No. 299,649 to Keep, et al. exemplify the use of electric switches connected to the side rails, headboard, and footboard of the bed to detect the movement of a patient attempting to climb over them.

U.S. Pat. No. 3,991,414 to Moran discloses the use of a mercury switch housed in a cartridge which is mounted on a portion of a bed frame for pivotal movement in response to the deflection of two deflection elements which move in accordance with the shift in weight of a patient who is changing positions on or is departing from the bed. A patient leaving the bed removes his weight therefrom to permit pivotal movement of the mercury switch. Such pivotal movement alters the position of the mercury contained within the switch cartridge so that the mercury comes into contact with the two spaced-apart switch electrodes to close the switch and produce an electric signal indicating that the patient has left the bed.

A prime example of a more evolved device is shown and described in U.S. Pat. No. 4,536,755 to Holzgang et al. The device mounts to the leg of a patient and detects through movement of mercury within an internal switch whether the patient has arisen to an ambulatory position from a reclined position, generating an alarm if he has done so.

A more recent invention described in U.S. Pat No. 5,751,214 assigned to Alert Care Inc. has tried to improve this kind of patient position monitoring device, but still came short. This last invention required an external device. As such it was not autonomous and needed to be wired to the door, the bed or the wall depending on its application. In addition, such prior art system could not detect impacts. The only function of the device was the detection of the patient movement when it interrupted the connection with the external device. Moreover, one of the main shortcomings of prior autonomous device was their low energy efficiency. In addition to sometime containing hazardous material such as mercury, previous devices were very energy consuming and often required battery replacements.

Although patient activity monitoring devices are well known, prior art models have featured significant shortcomings.

The primary drawback of these prior devices is their limited use and their short lifetime. Prior devices, such as mattresses for detecting whether the patient is still in bed or on a chair get damaged very easily. The patient monitoring device is a device which is used extensively due to ongoing requirement for the monitoring of the patient.

Another drawback of previous devices is their energy consumption. Such limitation mostly applies for devices attached to the leg of the patient as these devices normally are not wired to a power source. As such, the portable devices strapped to the patient leg tend to consume lots of battery power over time resulting in frequent battery changes. These devices are often used to monitor the patient during resting time such as when the patient is sleeping. Consequently, the device typically should not require active battery consumption

Accordingly, there is a need for patient activity monitoring device that is suitably designed for sustained use and which is generally designed in a way that mitigates sonic of the above-mentioned limitations.

### SUMMARY OF THE INVENTION

Accordingly, the present invention has been made to mitigate at least some of the above-mentioned shortcoming of the prior art by providing a system that is easy to operate and autonomous and more energy efficient.

As such, a patient safety position monitoring device and a method for monitoring a patient are disclosed. The device includes a handheld, portable housing comprising a processing unit (e.g. a microcontroller), a gyroscope module (e.g., an accelerometer module having gyroscope functions), and an alarm system. In response to the gyroscope module, the processing unit activates the alarm system to indicate that the patient, typically its leg, has moved beyond a prescribed angular limit.

The gyroscope module is designed for sensing the position of the patient, such as whether the patient is attempting to arise from a prone position. For its part, the processing unit, such as a microcontroller, is programmed to alert the care attendant of the various patient movements. The processing unit is also able to determine the angle of the patient through various sensors, such as the gyroscope module, and compare whether the preset angular limit has been reached.

The device in accordance with the principles of the present invention generally comprises three main components, the housing box, the securing means and the electronic components.

The housing box is generally designed to house and protect the electronic components from any hazard such as but not limited to hard impacts created from the falling of the patient, dust, liquids and other potential impacts. The housing box is generally made from polymer composite but could as well be made from any standard rigid components like metals or plastics.

The electronic components typically comprise the processing unit, the gyroscope module, and the alarm system. In addition, in an embodiment comprising an accelerometer module, the device generally comprises two potentiometers enabling the adjustment of the sensitivity of the accelerometer module responsible for the detection of the various shocks. The potentiometers are also generally used to adjust the sensitivity of the gyroscope function to the variation of the leg position around the three axes (X, Y, Z) thereby allowing some level of tolerance to the angular limit and preventing the triggering of the alarm upon insignificant movements of the patient leg.

The securing means is generally some kind of strap made from fabric, polymer or some mixture of polymer and fabric. The strap is fixedly attached to the housing box and used to secure the housing box to the patient leg.

In addition, in accordance with an aspect of this invention, as opposed to previous prior devices, the present device is self reliable. As such, the electronic components do not require communication with external sensors in order to determine the patient position. Such independent functioning is highly desired for many smaller care establishments which have limited means and still requires providing a high level of care to the patient. As such the use of a unitary device allows for better cost efficiencies.

In addition to alerting the attendant upon changes regarding the patient positioning beyond a specifics limit, the device equipped with an accelerometer module has the ability to detect impact. As such where a patient would simply roll from his bed to fall on the floor without any triggering of the alarm system due to no variation in the leg position beyond the preset limit, the device would still sense the impact generated from the falling of the patient and immediately trigger an alarm.

Furthermore, according to another aspect of the present invention, the device is generally able to, in addition to alerting the attendant via a sound transmission; communicate to a monitoring station in the care establishment using radio frequencies. Such embodiment would allow the device to alert an attendant located in a monitoring station far from the patient and allow for quicker interventions.

The method in accordance with the principles of the present invention generally comprises three main steps, installing the device on the patient, monitoring the patient position via the use of a portable device and comparing the monitored position against preset values.

More importantly, the present method for monitoring the patient position generally uses a. wireless device comprising a gyroscope module typically an accelerometer having gyroscope functions. As such, the present method for monitoring the patient position is different from prior art method requiring the use of wired devices.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### BRIEF DESCRIPTION OF THE DRAWINGS

FIG. 1 is a perspective view of a patient safety position monitoring device in accordance with the principles of the present invention.

FIG. 2 is a perspective view of the patient safety position monitoring device of FIG. 1, mounted to the leg of a patient, and with the housing box removed from the receptacle.

FIG. 3 is a side view of a patient leg having secured thereto a patient safety position monitoring device of FIG. 1.

FIG. 4 is another side view of a patient leg having secured thereto a patient safety position monitoring device of FIG. 1.

FIGS. 5-8 are a series of cut-away views showing how the housing box is inserted into the mounting receptacle, locked therein, and removed therefrom.

FIG. 9 is an illustration of a possible display of the electronic components.

FIG. 10 is a flowchart illustrating the process followed by the software of the device.

FIG. 11 is a diagram of an exemplary electronic circuit for the electronic components.

### DETAILED DESCRIPTION OF THE PREFERRED EMBODIMENT

A novel device and method for monitoring a patient position will be described hereinafter. Although the invention is described in terms of specific embodiments, it is understood that the embodiments described herein are by way of example only and that the scope of the invention is not intended to be limited thereby.

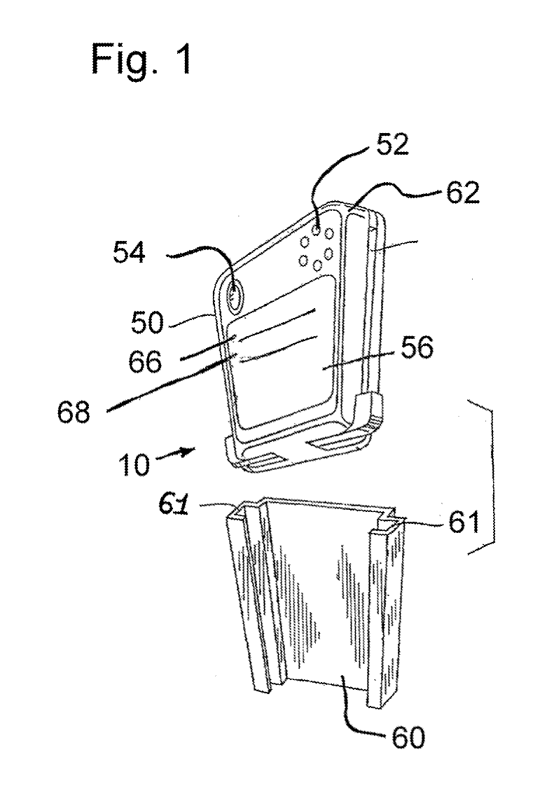

In the present embodiment, referring to FIG. 1, the patient position monitoring device 10 is designed to alert a care attendant of any patient movement beyond a prescribed threshold angular limit. The patient position monitoring device 10 comprises three main components, the housing box 50, the electronic module contained therein, and the receiving means 60.

The housing box module 50 is generally of prismatic shape, preferably small enough in size to be hand held. The housing box 50 is designed to house and protect the electronic components from any hazard such as to hard impacts created from the falling of the patient. In the present embodiment, the housing box 50 comprises a solid case 62, a key enable locking switch 54 and a display screen 56,

Still referring to FIG. 1, the locking switch 54 can be activated using a key. The locking switch 54 is used to reset the device upon activation as such it can reset both the impact alert and the movement alert. The key it typically kept by the attendant in charge of the patient. Such mechanism also prevents the patient from accidentally or voluntarily turning off the incident alert. The screen display 56 generally displays the angle 66 of the device and the chosen settings 68. In addition, upon activation of the alarm system, the display screen 56 will be backlighting further indicating the alert.

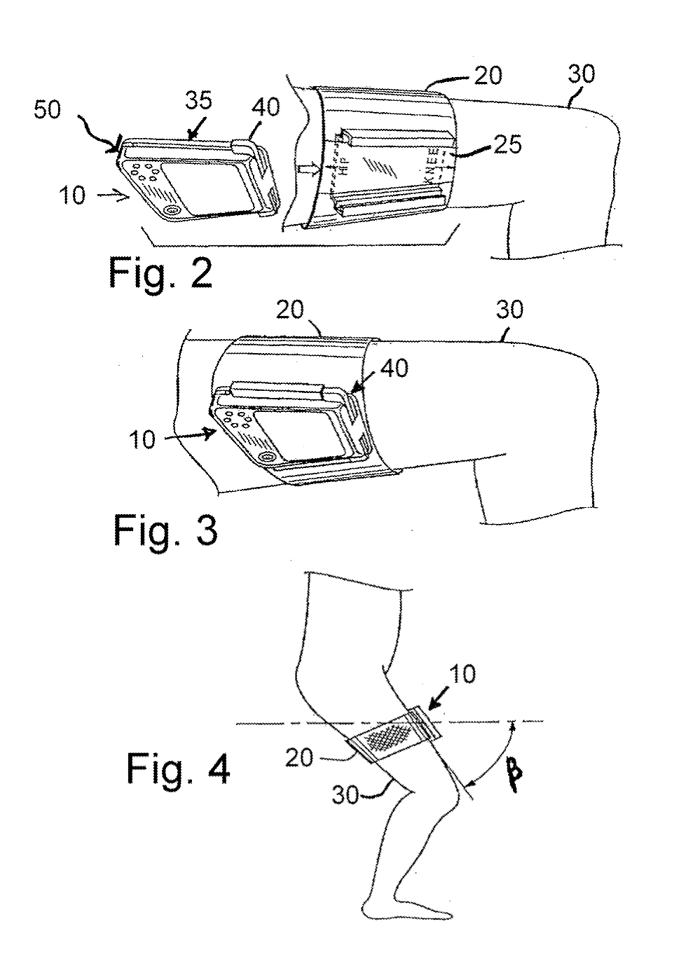

Now referring to FIGS. 2 to 4, a series of diagrams showing how the device 10 is used for monitoring the ambulatory position of a patient through internal sensors such as the gyroscope and accelerometer modules (not shown), Securing means 20, generally in the form of a strap made from either solid or elastic material, allow for the secure mounting of the housing box to the patient leg 70. For instance, the securing means 20 is a thigh clasp such as an elastic cloth and which encircles a patient's thigh 30. In FIG. 2 the housing unit 50 is shown being inserted into the securing means receptacle 25 and in FIG. 3 the unit is locked in place.

On a continuous basis, a processing unit monitors the angle of the patient legs.

For instance, when the housing box 50 is at roughly the horizontal angle, the gyroscope module (not shown) will indicate a 180 degree angular value. The processing unit (not shown) will process whether or not the preset angular limit has been breached and trigger or not the alarm as the case may be. In the case illustrated in FIG. 3, the limit will probably never have been reached and no alarm will be triggered. If as shown in FIG. 4, the patient rises from this horizontal position past a predetermined angle, the processing unit (not shown) will activate the alarm system (not shown) which will further emit an intermittent sound. In cases where the device is further equipped with a radio transmitter or a telecommunication module, the processing unit in response will trigger the alarm system which will transmit a signal indicating the patient is in movement and has breached the preset angular limit.

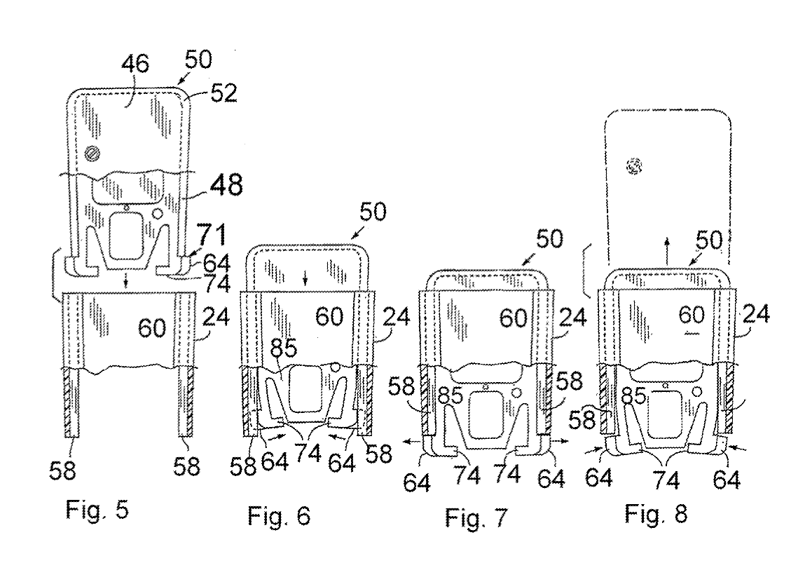

Now referring to FIGS. 5 to 8, a series of cut-away views are showing how the housing box 50 is inserted into mounting receptacle 24, locked therein, and removed therefrom. Referring to FIGS. 1 and 5, housing box includes a centerpiece 46 having a pair of resilient arms 48 that each taper inward and end in a catch 71. Each arm is notched between a top end 52 and a bottom end 74. The mounting receptacle includes a surface 60 supporting a pair of spaced-apart rails 58 that taper inward toward each other. The rails are substantially the length of the notched portion of the arms and are adapted to engage housing box 50 in slots 61 (FIG. 1) defined by the arms 48 and the front side 85 and rear side 62 of the housing. Catch 71 includes a tab 64 extending from the bottom arm end 74 for enabling one to press the arms 48 inward.

Referring to FIG. 6, the housing box 50 is shown slidingly engaged with rails 58 of mounting receptacle 24. Because of the tapering of the rails. The arms 48 and especially their bottom ends 74 frictionally engage the rails as the housing box 50 is inserted into the mounting receptacle. The tapering of the rails deflects catches 71 inwardly. But the catches are biased by the resiliency of the arms to press outward against the rails.

FIG. 7 shows the housing box 50 securely locked into place within mounting receptacle 24. As the bottom ends 74 of the arms 48 slide past the bottom ends of rails 58. The bottom arm ends press outward to engage the bottom rail ends. Similarly, the top arm ends 52 engage the top rail ends to prevent the housing box from sliding further relative to receptacle 24.

Now referring to FIG. 8, it is shown how the housing box 50 is removed from the mounting receptacle 24. Tabs 64 of catches 71 aresimply pressed inwardly to disengage the bottom arm ends 74 from the bottom ends of rails 58 and the unit slid outward along the rails 58,

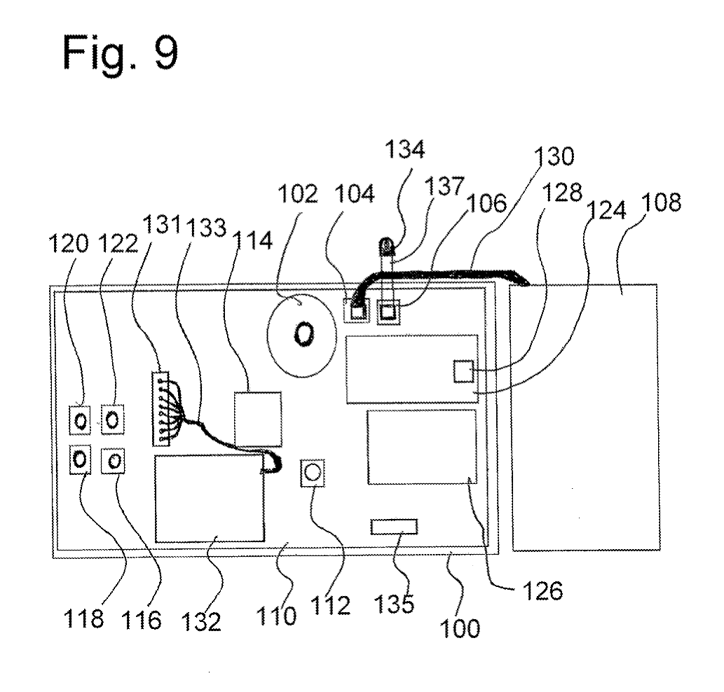

Now referring to FIG. 9, in the present embodiment, the back end 100 of the housing box 50 has the electronic module mounted therein. The electronic module typically comprises a main board 110, the processing unit 114, the gyroscope module, preferably an accelerometer module having gyroscope functions 124, four potentiometers 116, 118, 120 and 122, a display screen 132, a reset button 112, a display connector 131, a display connection wire 133, an IDE, connector 135, the alarm system 102, typically a small sound-speaker, and a battery 108. The power cord is generally connected to the battery plug 104, beside which may be found the interrupter plug 106 whereby the interrupter wire 137 is connected. The interrupter wire 137 is typically connecting the key switch 134 which is trigger of the reset function.

In another embodiment, the battery 108 and power supply cord 130 are connected to a charging module 124 comprising a USE connection 128. Such configuration allow for the charging of the device using a commercially available USE charger. In addition, in another embodiment, the circuit may be configured to have the power supply mediated by the accelerometer module. As such, unless there is some movement or impact sensed, the device power will be shut down thereby preventing any useless power consumption.

It is possible to have the device 110 configured to only utilise the power when a movement is sensed by the accelerometer module 126. As such in times when no movement is sensed, the device is in a kind of hibernation. This state of hibernation is quickly reversed upon sensing movement from the accelerometer module 126. This configuration will generally allow the battery to last substantially longer. Accordingly, using the device for patients who remain in bed without triggering the alert will result in mitigating the shortcoming of high energy consumption devices.

In the present embodiment, the alarm system 102 generally comprises a sound emitter which is typically triggered by the processing unit 114 when either the angle has reached the threshold limit or when an impact has been detected by the accelerometer module 126. The sound will be emitted on an intermittent basis wherein the triggering event was the breach of the preset angle. Whereas, in cases where an impact has been detected, the sound will be emitted as a continuous sound.

In another embodiment, the patient position monitoring device can further comprise an emitter, typically a radio frequency emitter, such as, a TXA33 transmitter of 433 MHz able to emit over more than 5000 feet. The radio transmitter is allowing the patient position monitoring device to alert an attendant located in another room via a portable receiver or where a central monitoring station is available, directly to the monitoring station. Such monitoring station could be located elsewhere in the care giving establishment through the use of telecommunication. According to another embodiment, the telecommunication module could typically be cell phone based transmitter.

Due to the nature of the device and its sensitivity to small body movement, the present device 10 has been designed to allow for the presetting of certain critical angles which will ultimately trigger the alarm system 102. The critical angle and the intensity of the shock required to trigger the alarm is set using the four potentometers 116, 118, 120 and 122 or commonly referred to as setting buttons.

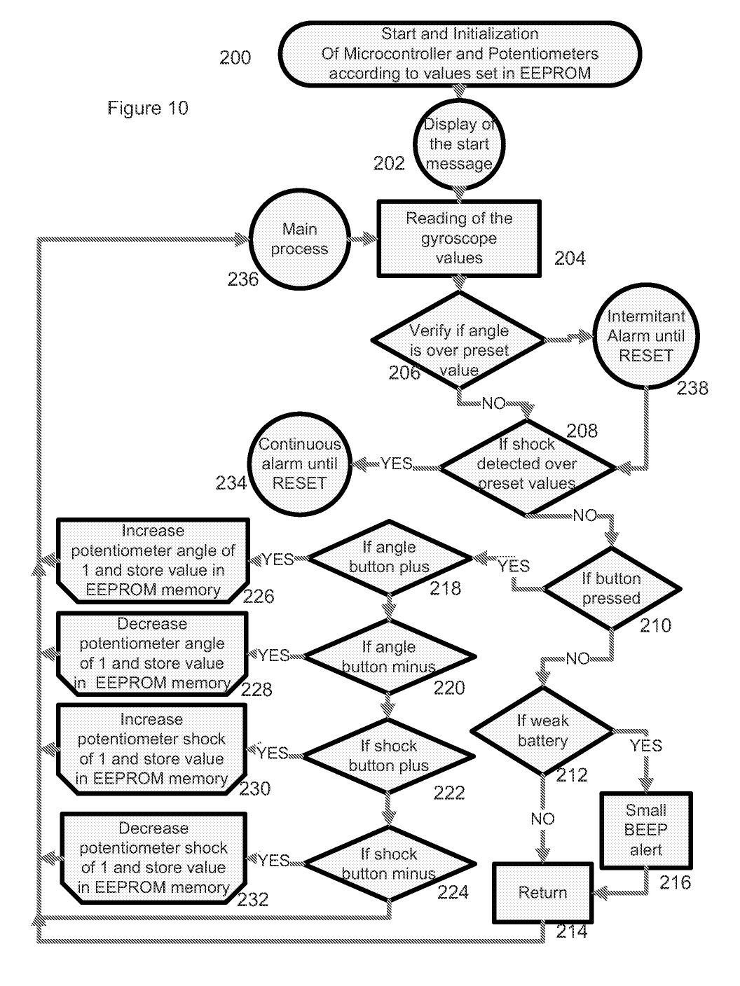

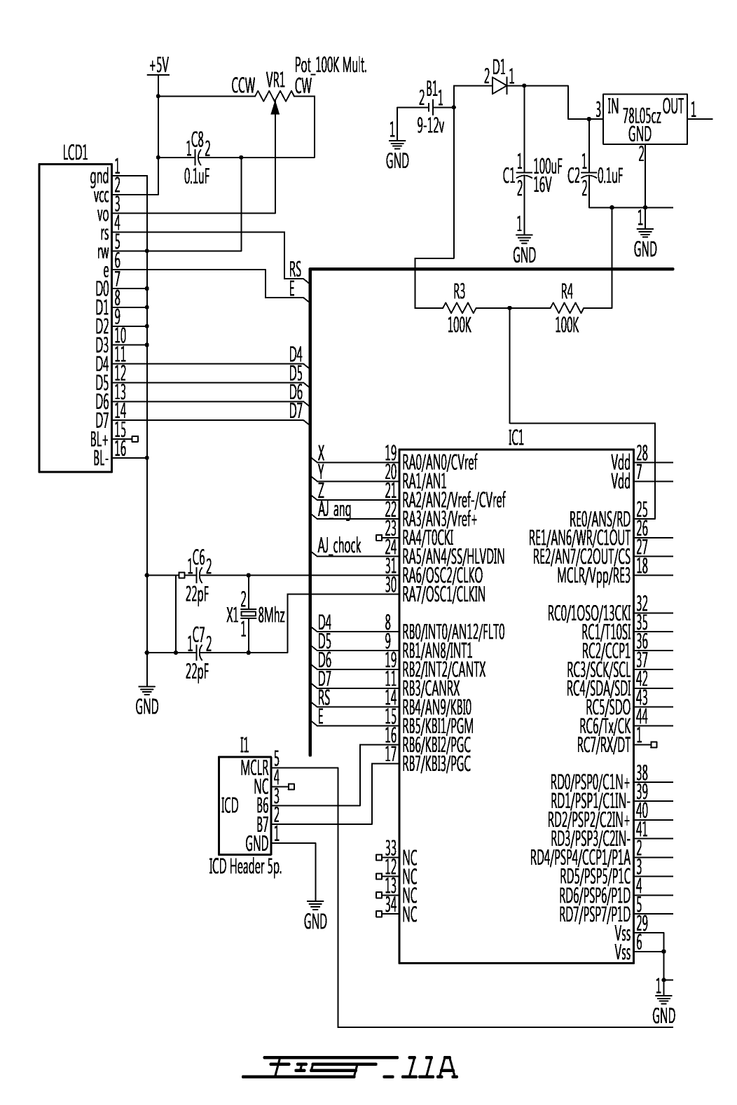

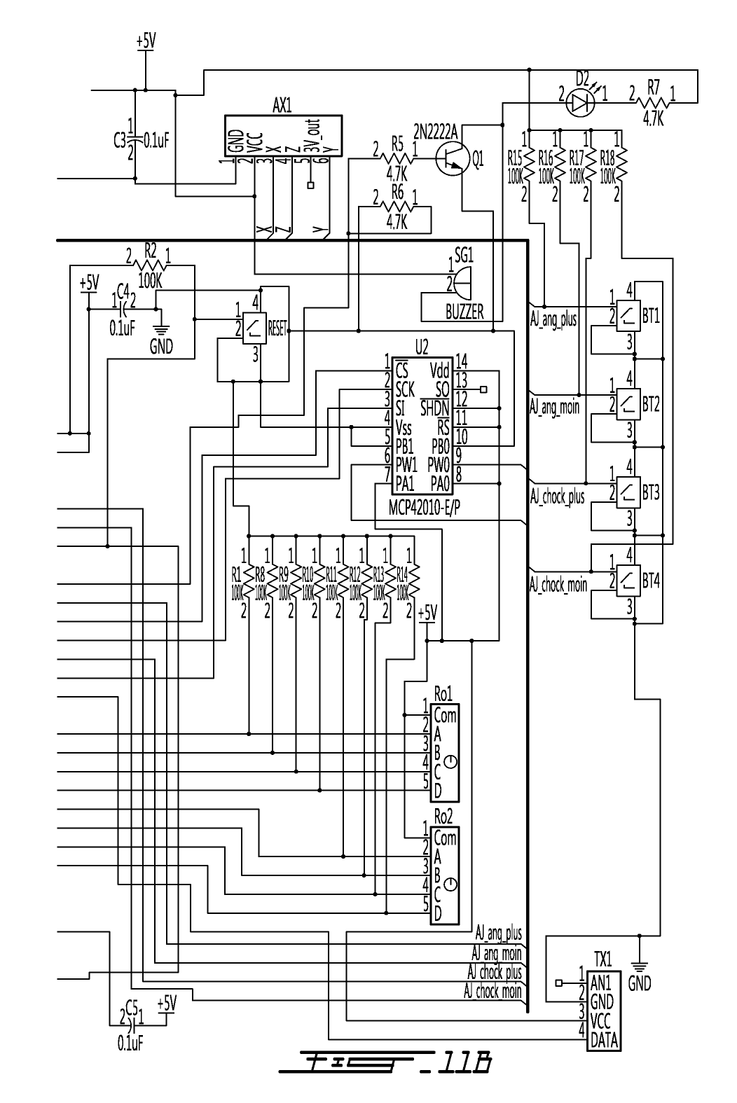

Now referring to FIG. 10, an example of the process for the ongoing monitoring of the angle and for insuring that no impact has been detected is illustrated through a flowchart. The present software enabled process starts with the initialization step 200 which displays the start message 202. The software will then verify the readings of the gyroscope module 204 followed by a comparison process to see if the angular values are over the preset values 206. If the preset values have been reached, the YES answer will trigger an intermittent alarm 238 until the RESET function is triggered and continue the process. If the preset values have not been reached, the NO answer will continue the process. The next step in the main process requires verifying if a shock has been detected, if the accelerometer value is over the preset values 208. If a shock over the preset values has been detected, the process will return a YES response and trigger a continuous alarm until the RESET 234 function is triggered. If no shock over the preset values has been detected the process will return a NO response and continue the main process. The next step requires the process to verify if a button has been pressed 201. If a button has been pressed, the device will first identify which button has been pressed 218, 220, 222, 224, and result in the corresponding action of decreasing or increasing the angle or the shock memory value 226, 228, 230, 232. In cases no button has been pressed, the NO answer will allow the process to continue. The next step in the process requires the process to verify if the battery is weak 212. If a yes response is returned, a small BEEP alert will be triggered 216. If the battery has not been detected as weak the process continues to the next step, the return function 214, wherein the process is directed to the main process 236 and repeat itself.

### EXAMPLE

An example of a functioning circuit is illustrated in FIG. 11. The processing unit X (model PIC dip40-18F4685), represents the core of the system as it is the main component for managing the gyroscope and the accelerometer modules and ultimately triggering the alarm system. In this embodiment, the AX1 is the detector for the three axes (X, Y, Z). The A3 port is generally used for reading of the digital potentiometer used for presetting the desired tolerance angle. The A5 port is generally used for the reading of the digital potentiometer used for presetting the desired shock sensitivity. The A6 and A7 ports are used for the 8 MHz Crystal Oscillator (CO). The CO port is the exit for the activation of the micro sound speaker. The exit for the micro sound speaker is mediated via the R5 and R6 (resistance of ¼ W 5% SMD type 1206). The transistor Q1 is the NPN standard which is used as reference for the breach of the preset angle or the intensity of the shock. The radial electrolytic capacitor C1 allows the reading of the button for the increase of the potentiometer which gives the desired angle. The ceramic capacitor C2 is used for the selection of the digital potentiometer. The C3 and C5 capacitor are used for SPI communication of the digital potentiometer. The capacitor C4 allows the reading of the button for reducing the potentiometer related to the wanted angle. The capacitor C6 sends the signal to the series RS232 to 4700 Bps for the emission of the alarm signal. The D port is used for the reading of the rotative contractor for the selection of the module number. The rotative contractors Ro1 and Ro2 are binary rotative connectors used for giving a number between 0 and 255 to the circuit for the transmission of a unique code to the receptor during an alarm event. As such, the device is able to code a different reference for each patient. R1, R8 to R14 give a negative signal where no commutator has been selected. The MCP42010-E/P are 10K digital potentiometers allowing the selection of the desired values for the angles and the shocks.

R6 is used for transmitting a negative signal to the transistor in order to prevent the alarm during the initialization of the mike, R15 and R18 give a positive signal by default to the adjustment button. BT1 to BT4 are adjustment buttons for the potentiometers. AX1 is the three axes (X,Y,Z) accelerating module for both angle and shocks. L1 is the connector for programming the processing unit. Every capacitors are used for the filtration of the DC current. The U1 is a regulator 78L05 5v 100 mA. D1 is a diode used for preventing an inverted connection of the battery which would ultimately result in the destruction of the circuits. VR1 is a potentiometer for the adjustment of the density of the LCD display when in use. BZ1 is a 5V Buzzer used for the emission of the sound alarm. PROG is a four pin header used for the external programming of the processing unit. As such, using this four pin header, it is possible to program the processing unit from an external terminal. TX1 (TXA33) is a signal transmitter which emits a signal to a programmed receptor. Such programmed receptor is optional and already commercially available. The RESET button is a button for the reinitialization of the device circuit. U1 is a 5V 200 mA regulator.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

1. A patient position monitoring device comprising:
a housing box;
a securing means;
an alarm system;
an electronic circuit;
wherein the electronic circuit comprises a gyroscope function and a (processing, unit configured to detect the position of a patient.

2. A patient position monitoring device as claimed in claim 1 wherein the gyroscope is an accelerometer module.

3. A patient position monitoring device as claimed in claim 1 wherein the alarm system is a sound speaker.

4. A patient position monitoring device as claimed in claim 1 wherein the alarm system is a radio transmitter, the transmitter generating a binary signal for indicating whether the patient has triggered the alert.

5. A patient position monitoring device as claimed in claim 1 wherein the alarm system further comprises a radio transmitter.

6. A patient position monitoring device as claimed in claim 1 wherein the electronic circuit further comprises at least one potentiometer for controlling the sensitivity of the gyroscope module.

7. A patient position monitoring device as claimed in claim 1 wherein the device further comprises a reset function for disabling the alarm system.

8. A patient position monitoring device as claimed in claim 7 wherein the reset function is activated by a lock and key mechanism.

9. A patient position monitoring device as claimed in claim 1 further comprising a touch screen.

10. A patient position monitoring device as claimed in claim 1 wherein the gyroscope module is configured to allow the presetting of threshold angular values.

11. A patient position monitoring device as claimed in claim 2 wherein the accelerometer module is configured to allow the presetting of threshold shock values.

12. A patient position monitoring device as claimed in claim 1 wherein the power supply is mediated by the gyroscope module and configured to allow the power upon detection of an angle variation.

13. A patient position monitoring device as claimed in claim 2 wherein the power supply is mediated by the accelerometer module and configured to allow the power upon detection of impacts.

14. A patient monitoring device as claimed in claim 1 further comprising a buckle securing means.

15. A method for monitoring a patient position comprising:
mounting a monitoring device to a patient leg;
starting the device;
monitoring angular values of a gyroscope module of the device;
comparing the angular values with preset angular values;
triggering an alarm when the angular values have reached the preset angular values.

16. A method for monitoring a patient position as claimed in claim 15, the method further comprising:
monitoring accelerometer values of an accelerometer module of the device;
comparing impact values with preset accelerometer values;
triggering an alarm when the impact values have reached the preset accelerometer values.

17. A method for monitoring a patient position as claimed in claim 15 wherein the alarm will result in a transmission of a radio signal indicating that the patient has breached preset values.
